(19) United States
(12) Patent Application Publication
(10) Pub. No.: US 2026/0301855 A1
(43) Pub. Date: Oct. 1, 2026

(54) METHOD FOR DERIVING CHARACTERISTIC DNA PARAMETERS PARAMETER-FREE FROM RECOGNITION PHYSICS PRINCIPLES

(71) Applicant: Jonathan Washburn, Austin, TX (US)

(72) Inventor: Jonathan Washburn, Austin, TX (US)

(21) Appl. No.: 19/629,715

(22) Filed: Mar. 26, 2026

Related U.S. Application Data
(60) Provisional application No. 63/779,042, filed on Mar. 27, 2025.

(51) Int. Cl. G16B 15/10; G16B 5/00; G16B 30/00; G16B 50/30

(52) U.S. Cl. CPC G16B 15/10; G16B 5/00; G16B 30/00; G16B 50/30

(57) ABSTRACT

A computer-implemented method, system, and non-transitory computer-readable medium derive characteristic physical parameters associated with deoxyribonucleic acid (DNA) from Recognition Physics principles without using empirically fitted DNA-specific coefficients as inputs to the core derivation. A target DNA parameter is identified, classified, and assigned to a derivation pathway including cascade indexing, geometric scaling, or efficiency modulation. In preferred embodiments, the system derives one or more of a characteristic DNA length parameter X_DNA, a helical pitch parameter P0, a coherence-energy parameter E_coh, a base-pair stabilization parameter E_bp, and a characteristic rate parameter R0. The system generates a derived numerical value and a derivation trace and uses the derived numerical value in DNA design, construct selection, sequence screening, simulation initialization, or validation workflows. Sequence-specific, analog-specific, system, database, and application programming interface embodiments are also disclosed.

### CROSS-REFERENCE TO RELATED APPLICATIONS

This application claims the benefit of U.S. Provisional Patent Application No. 63/779,042, filed on Mar. 27, 2025, and titled “Method for Deriving Characteristic DNA Parameters Parameter-Free from Recognition Physics Principles.”

### BACKGROUND OF THE INVENTION

The present disclosure relates generally to computational molecular biology, biophysics, nucleic-acid engineering, synthetic biology, and computer-implemented parameter derivation systems. More particularly, the present disclosure relates to methods, systems, and non-transitory computer-readable media for deriving characteristic physical parameters associated with deoxyribonucleic acid (DNA) from Recognition Physics principles using universal physical constants, forced Recognition Science constants, and defined structural relationships, without requiring empirically fitted DNA-specific parameters as inputs to the core derivation.

DNA exhibits a number of characteristic physical parameters that are central to molecular structure, stability, dynamics, recognition, transcription, and engineering. Such parameters can include, by way of example and not limitation, inter-strand spacing, groove-related dimensions, helical pitch, base-pair stabilization energies, coherence-associated energy scales, rate scales associated with transcription-related processes, and higher-order derived quantities that depend on one or more of the foregoing. These parameters are important in both natural biological analysis and synthetic or engineered nucleic-acid systems.

Conventionally, many DNA-related parameters are obtained or approximated through experimental measurement, empirical fitting, molecular simulation, quantum-chemical estimation, statistical modeling, or combinations thereof. For example, structural parameters may be inferred from X-ray diffraction, cryo-electron microscopy, nuclear magnetic resonance, or structural database analysis. Energetic parameters may be estimated from calorimetry, melting studies, nearest-neighbor thermodynamic models, electronic structure calculations, or empirical sequence datasets. Kinetic or transcription-related quantities may be inferred from biochemical assays, polymerase studies, fluorescence measurements, or fitted rate models. While these techniques can be useful, they generally do not provide a unified first-principles derivation framework for multiple characteristic DNA parameters from a common physical architecture.

Existing approaches also tend to treat different classes of DNA parameters separately. Structural geometry, energetic stabilization, coherence-related behavior, and kinetic scaling are commonly modeled using distinct assumptions, distinct datasets, and distinct fitting procedures. As a result, even when useful values are obtained, the values are often not derived as parts of a single internally linked framework. This fragmentation can make it difficult to generate consistent parameter sets for downstream DNA modeling, design, validation, synthesis planning, or nucleic-acid engineering workflows.

In many instances, prior methods depend upon empirically fitted constants that are specific to DNA or to a subclass of DNA systems. Such fitted parameters may be sensitive to the source dataset, measurement conditions, ionic environment, hydration state, sequence distribution, experimental protocol, or model assumptions used to generate them. Although fitted models may achieve acceptable predictive performance within the regimes for which they were tuned, they may not readily generalize outside those regimes, and they may not explain why a given parameter takes the observed magnitude from a more foundational physical standpoint.

A further limitation of conventional DNA parameter estimation techniques is that they often do not produce a derivation trace that can be readily audited, reproduced, or reused across multiple computational environments. In some workflows, a user may receive only a final numerical output, a black-box simulation result, or a parameter value extracted from a reference table, without a unified explanation of how the value was generated from a first-principles framework. This can reduce transparency and can hinder integration into high-confidence engineering systems, parameter libraries, automated design tools, or verification pipelines.

There is accordingly a need for a unified framework capable of deriving one or more characteristic DNA parameters from foundational physical principles rather than from empirically fitted DNA-specific inputs. There is also a need for such a framework to support multiple classes of outputs, including geometric outputs, energetic outputs, rate outputs, and dependent or extended outputs, while maintaining internal consistency among the derived values. There is further a need for such a framework to be implementable in software, network services, databases, validation systems, and design platforms so that derived values can be generated, stored, transmitted, compared, and applied in practical DNA-related workflows.

“Recognition Physics” or “Recognition Science,” as used herein, refers to a deterministic mathematical and physical framework in which discrete recognition-cost relationships and closure constraints define one or more universal constants and rule-based selection criteria used to derive characteristic parameters of physical systems. Unless expressly recited in a claim, statements describing Recognition Science as a foundational or underlying architecture are provided as explanatory context and do not limit the scope of the claimed embodiments.

Recognition Science and Recognition Physics provide a foundational framework in which certain physical quantities can be derived from first principles using a constrained cost architecture, forced constants, and scale relations. In preferred embodiments relevant to the present disclosure, a characteristic optimization constant X_opt is defined as X_opt=phi/pi, where phi=(1+sqrt(5))/2. In further preferred embodiments, a cost function is defined as J(x)=0.5*(x+1/x)−1. In additional preferred embodiments, scale cascades may be expressed from Planck-scale anchor quantities, including a length cascade r_n=L_Planck*(X_opt)\hat{ }n and an energy cascade E_n=E_Planck*(X_opt)\hat{ }n, where L_Planck is Planck length and E_Planck is Planck energy. These relationships provide a basis from which DNA-relevant characteristic parameters can be derived in a structured and reproducible manner.

In one aspect, the present disclosure recognizes that characteristic DNA quantities can be derived through one or more pathway classes selected according to the type of target parameter. Such pathway classes can include, for example, cascade indexing, geometric scaling, and efficiency modulation. In a cascade indexing embodiment, a target parameter may be generated by selecting an index n relative to a Planck-scale anchor quantity and applying X_opt scaling. In a geometric scaling embodiment, a target parameter may be derived from another derived parameter through one or more geometry-related factors, such as phi-based closure or self-similar scaling. In an efficiency modulation embodiment, a target parameter may be produced by modulating a source parameter according to an X_opt factor, a multiplicity factor, a sequence factor, or another discrete structural factor.

A further need exists for a derivation framework that can generate not merely isolated outputs but a linked family of DNA parameters in which one derived quantity supports or constrains another. By way of example, a derived characteristic distance can support a helical pitch derivation; a derived coherence-energy scale can support a base-pair stabilization energy derivation; and structural and energetic outputs can further support a characteristic rate derivation. A unified linked framework is advantageous because it allows downstream users to obtain a consistent parameter set rather than a patchwork of unrelated estimates.

A further need exists for a derivation system that can distinguish between core derivation inputs and optional post-derivation comparison references. In preferred embodiments of the present disclosure, the core calculation is parameter-free in the sense that empirically fitted DNA-specific constants are not required as inputs to generate the target parameter. However, experimentally observed or literature-reported values may still be used after derivation for validation, benchmarking, ranking, confidence reporting, or comparison tables. This distinction improves clarity and helps preserve a first-principles character for the derivation itself while still allowing the practical use of external references.

There is also a need for a derivation architecture that can support sequence-specific or composition-specific embodiments without collapsing into a purely empirical model. For example, in some embodiments, a target DNA parameter may depend in part on a discrete structural multiplicity or composition factor based on the number of hydrogen bonds associated with adenine-thymine and cytosine-guanine base pairs. A system that can incorporate such structural factors while avoiding empirically fitted DNA-specific constants provides a useful middle ground between rigid abstraction and purely data-fit parameterization.

There is additionally a need for a derivation system that can support nucleic-acid analogs and variant systems, including but not limited to ribonucleic acid (RNA), peptide nucleic acid (PNA), xDNA systems, Hachimoji systems, modified backbones, engineered base sets, and hybrid constructs, while preserving a common first-principles framework. In such embodiments, one or more derivation inputs or selection rules may be modified to reflect differing geometries, multiplicities, stability criteria, interaction classes, or structural constraints associated with the analog or variant system under consideration.

Another limitation in the prior art is that many DNA modeling approaches are not well integrated with practical engineering workflows. A researcher or engineer may need to derive or select DNA-relevant parameters for use in sequence design, construct design, coherence-first design, transcription analysis, simulation initialization, screening of candidate systems, reporting through application programming interfaces, storage in parameter libraries, or physical synthesis and validation. Existing tools often require the manual gathering of unrelated values from the literature or from different software packages. A first-principles derivation system that directly produces such parameters in a machine-readable and workflow-ready form would therefore be advantageous.

There is further a need for systems that can generate characteristic DNA parameters together with derivation traces, intermediate values, symbolic equations, proof references, validation tables, exportable records, and downstream-use tags. Such enriched outputs can improve auditability, reproducibility, and reuse across research, patenting, software implementation, laboratory planning, design automation, and quality-control settings.

The present disclosure addresses these and other needs by providing methods, systems, and non-transitory computer-readable media for deriving characteristic DNA parameters parameter-free from Recognition Physics principles. In preferred embodiments, the disclosure provides a framework for deriving a family of canonical DNA outputs including a characteristic DNA length parameter X_DNA, a helical pitch parameter P0, a coherence-energy parameter E_coh, a base-pair stabilization parameter E_bp, and a characteristic rate parameter R0. In additional embodiments, the disclosure provides methods for deriving one or more extended parameters, sequence-specific variants, analog-specific variants, and downstream engineering parameters based on the same foundational framework.

The present disclosure further addresses practical application by providing embodiments in which the derived parameters are used in computational pipelines, parameter databases, network-accessible derivation services, coherence-first design systems, DNA construct definition workflows, simulation initialization systems, and validation platforms. In this manner, the disclosure provides not only a foundational derivation methodology but also an examiner-ready practical architecture for generating and using characteristic DNA parameters in technically concrete contexts.

### BRIEF SUMMARY OF THE INVENTION

The present disclosure provides methods, systems, and non-transitory computer-readable media for deriving characteristic physical parameters associated with deoxyribonucleic acid (DNA) from Recognition Physics principles without requiring empirically fitted DNA-specific parameters as inputs to the core derivation. In preferred embodiments, the disclosed framework uses one or more universal physical constants, one or more forced Recognition Science constants, and one or more defined structural relationships to produce reproducible DNA-relevant outputs from a unified first-principles architecture.

In preferred embodiments, the disclosure provides a computer-implemented method in which a target characteristic DNA parameter is identified, a derivation pathway is selected according to the type of target parameter, one or more permitted constants or structural inputs are applied, and a derived numerical value is generated and output. In some embodiments, the target parameter is a length-type parameter, an energy-type parameter, a rate-type parameter, a geometry-type parameter, a multiplicity-dependent parameter, or an extended parameter derived from one or more previously derived parameters.

In preferred embodiments, the disclosure provides a canonical five-parameter derivation framework for DNA. The canonical framework can include derivation of a characteristic DNA length parameter X_DNA, a characteristic helical pitch parameter P0, a characteristic coherence-energy parameter E_coh, a characteristic base-pair stabilization parameter E_bp, and a characteristic rate parameter R0. In preferred embodiments, these parameters are not generated as isolated outputs but rather as a linked family in which one derived quantity can support, constrain, or inform one or more other derived quantities.

In one preferred embodiment, the characteristic DNA length parameter is derived according to X_DNA=L_Planck*(X_opt)\hat{ }(−90), where X_opt=phi/pi, phi=(1+sqrt(5))/2, and L_Planck is Planck length. In preferred embodiments, X_DNA corresponds to a characteristic DNA spatial scale and can be used as a base quantity for one or more downstream geometric derivations.

In one preferred embodiment, the characteristic helical pitch parameter is derived according to P0=X_DNA*(phi\hat{ }2). In preferred embodiments, P0 represents a characteristic DNA helical pitch scale and may be further used to derive or support one or more additional geometric or rate-related outputs. In some embodiments, the ratio P0/(X_DNA/4) defines a characteristic bases-per-turn quantity.

In one preferred embodiment, the characteristic coherence-energy parameter is derived according to E_coh=E_Planck*(X_opt)\hat{ }(101), where E_Planck is Planck energy. In preferred embodiments, E_coh represents a canonical coherence-associated energy scale for DNA and may be used directly or in scaled form in one or more downstream design, construct, or validation embodiments.

In one preferred embodiment, the characteristic base-pair stabilization parameter is derived according to E_bp=E_coh*X_opt*N_H, where N_H is a hydrogen-bond multiplicity parameter. In some embodiments, N_H is assigned a default value. In other embodiments, N_H is based on sequence composition, base-pair identity, local sequence windows, or another discrete structural multiplicity associated with the DNA system being evaluated.

In one preferred embodiment, the characteristic rate parameter is derived according to R0=(E_coh/h)*(P0/(X_DNA/4))*(2/(phi\hat{ }62)), where h is Planck's constant. In preferred embodiments, R0 represents a characteristic rate scale associated with DNA-related activity and may be reported in bases per second or in another suitable unit.

In some embodiments, the disclosed derivation architecture classifies target parameters into one or more pathway classes. Such pathway classes can include cascade indexing, geometric scaling, and efficiency modulation. In a cascade indexing embodiment, a target parameter is derived by selecting an index relative to a Planck-scale anchor quantity and applying X_opt scaling. In a geometric scaling embodiment, a target parameter is derived from another derived quantity according to one or more geometry-related scale factors. In an efficiency modulation embodiment, a target parameter is derived by applying an X_opt factor, a multiplicity factor, or another discrete structural factor to a source quantity.

In some embodiments, a derivation pathway is selected at least in part according to a Minimal Overhead principle or another rule-based selection architecture. In such embodiments, the system may determine one or more cascade indices, exponents, multiplicity factors, or geometric factors based on dimensionality, structural class, interaction type, analog type, stability considerations, target-parameter identity, or combinations thereof. In other embodiments, pathway selection may be implemented through a rule table, lookup structure, certified relation set, optimization routine, or predetermined parameter map.

In some embodiments, the disclosed methods derive multiple canonical outputs during a single execution. For example, the system can derive X_DNA, P0, E_coh, E_bp, and R0 in one computational session and can optionally generate one or more linked intermediate values, dependency chains, consistency checks, or downstream-use outputs based on the jointly derived parameter set.

In some embodiments, the disclosure provides sequence-specific derivation embodiments. For example, a base-pair stabilization parameter can be generated using a sequence-dependent hydrogen-bond multiplicity given by N_H=(2*#AT+3*#CG)/(#AT+#CG), where #AT represents the number of adenine-thymine base pairs and #CG represents the number of cytosine-guanine base pairs in a selected region, sequence, library, or window. In other embodiments, local, regional, global, averaged, weighted, or analog-specific multiplicity formulations may be used.

In some embodiments, the disclosure provides extended parameter derivations in addition to or instead of the canonical five outputs. Such extended parameters can include one or more geometry quantities, rates, rigidities, densities, coupling terms, normalized factors, stability indicators, coherence-level values, analog-specific values, or other quantities derived from one or more canonical outputs and one or more permitted structural relationships.

In some embodiments, the disclosure provides scaled coherence embodiments in which one or more higher-order coherence-associated quantities are derived from E_coh. For example, a coherence quantity E_n may be defined as E_n=n*E_coh, where n is an integer or another permitted scaling factor. In related embodiments, one or more scaled rate quantities may be derived from R0, including embodiments in which R=n*R0 or in which another scaling relationship is applied for a selected physical or engineering context.

In some embodiments, the disclosure provides practical computational implementations including stand-alone software, workstation-based systems, cloud-based systems, distributed systems, embedded systems, network services, and application programming interfaces. In preferred embodiments, the system can receive one or more target parameter identifiers and optional contextual inputs, perform one or more derivations, and return one or more numerical outputs together with associated equations, derivation traces, intermediate values, validation information, or exportable records.

In some embodiments, the disclosure provides systems including one or more processors, memory devices, storage devices, user interfaces, and communication interfaces configured to perform the disclosed derivations. In some embodiments, the system includes a target parameter identifier module, a pathway selector, a cascade-index selector, a calculator, a unit conversion module, a derivation trace generator, a validation module, and a storage or reporting module. In some embodiments, these modules are implemented in software. In other embodiments, one or more modules may be implemented in hardware, firmware, or combinations thereof.

In some embodiments, the disclosure provides one or more network-accessible derivation services. Such services can receive requests identifying a target parameter, analog type, sequence composition, output format, precision selection, or validation option, and can return one or more derived values and associated metadata in machine-readable form. In some embodiments, the service stores one or more parameter records in a database, parameter library, or versioned derivation repository.

In some embodiments, the disclosure provides validation and comparison functionality in which a derived parameter is compared to one or more reference values after derivation. In preferred embodiments, such comparison values are not required as inputs to the core first-principles derivation, but may be used for benchmarking, ranking, confidence reporting, display, export, or audit purposes. In some embodiments, the system generates a derivation trace together with a validation table, percent-difference output, literature comparison report, or consistency indicator.

In some embodiments, the disclosure provides downstream engineering embodiments in which one or more derived DNA parameters are used to configure, constrain, initialize, or validate another technical workflow. Such workflows can include DNA construct design, coherence-first design, simulator initialization, sequence screening, library generation, structural validation, parameter database generation, laboratory planning, synthesis selection, or functional performance assessment.

In some embodiments, the disclosure provides design embodiments in which one or more derived quantities are used to define or constrain a DNA construct, a DNA sequence set, a coherence-supporting molecular architecture, or a formal design representation. In some embodiments, the derived values are used in connection with a design tuple, a stability threshold, a geometry specification, a coherence-level target, a functional use target, or a validation routine.

In some embodiments, the disclosure provides embodiments directed to nucleic-acid analogs and variant systems, including but not limited to RNA, PNA, xDNA systems, Hachimoji systems, modified backbones, modified bases, hybrid nucleic-acid systems, and engineered nucleic-acid constructs. In such embodiments, one or more derivation rules, multiplicity rules, geometry factors, index-selection rules, or stability criteria may be modified while preserving a common first-principles architecture.

In some embodiments, the disclosure provides embodiments in which one or more physical constructs are selected, designed, synthesized, assembled, screened, or validated using one or more disclosed derived parameters. Such constructs can include natural or synthetic DNA structures, coherence-supporting constructs, functionalized constructs, hybrid constructs, or multi-component assemblies. In some embodiments, the constructs are evaluated for one or more of structural stability, energetic suitability, coherence compatibility, transfer capability, catalytic suitability, signaling suitability, or another technical performance criterion.

In some embodiments, the disclosure provides non-transitory computer-readable media storing instructions that, when executed by one or more processors, cause the one or more processors to perform any of the methods described herein. In some embodiments, the disclosure further provides systems and media configured to derive a single target parameter, a selected subset of parameters, or a full parameter family for a given DNA system, sequence, analog, or design context.

These and other features, aspects, embodiments, implementations, advantages, and applications of the present disclosure will become apparent from the following detailed description, the accompanying drawings, and the appended claims.

### BRIEF DESCRIPTION OF THE DRAWINGS

The foregoing and other objects, features, and advantages of the present disclosure will be more fully understood from the following description of illustrative embodiments, as taken in conjunction with the accompanying drawings.

FIG. 1 is a flowchart illustrating an example method for deriving one or more characteristic DNA parameters from Recognition Physics principles, including target-parameter identification, pathway selection, derivation, and output generation.

FIG. 2 is a block diagram illustrating an example derivation system configured to generate characteristic DNA parameters, the system including a target parameter identifier, a pathway selector, a cascade-index selector, a calculator, a derivation trace generator, a validation module, and a storage or reporting module.

FIG. 3 is a diagram illustrating an example dependency architecture among canonical derived DNA parameters, including a characteristic DNA length parameter X_DNA, a helical pitch parameter P0, a coherence-energy parameter E_coh, a base-pair stabilization parameter E_bp, and a characteristic rate parameter R0.

FIG. 4 is a flowchart illustrating an example pathway-selection architecture in which a target parameter is classified and assigned to a derivation pathway including cascade indexing, geometric scaling, efficiency modulation, or a related derivation pathway.

FIG. 5 is a diagram illustrating an example Minimal Overhead selection framework for determining one or more cascade indices, exponents, multiplicity values, or structural factors used in the derivation of one or more characteristic DNA parameters.

FIG. 6 is a diagram illustrating an example derivation pathway for a characteristic DNA length parameter X_DNA derived from a Planck-length anchor and an X_opt scaling relation.

FIG. 7 is a diagram illustrating an example derivation pathway for a characteristic helical pitch parameter P0 derived from X_DNA and a geometry-related scaling factor.

FIG. 8 is a diagram illustrating an example derivation pathway for a characteristic coherence-energy parameter E_coh derived from a Planck-energy anchor and an X_opt scaling relation.

FIG. 9 is a diagram illustrating an example derivation pathway for a characteristic base-pair stabilization parameter E_bp derived from E_coh, an X_opt factor, and a hydrogen-bond multiplicity parameter N_H.

FIG. 10 is a diagram illustrating an example derivation pathway for a characteristic rate parameter R0 derived from E_coh, a characteristic bases-per-turn relation, and a biological efficiency factor.

FIG. 11 is a block diagram illustrating an example computing device for implementing the derivation system of FIG. 2.

It is to be understood that the drawings are provided for purposes of illustration and description and are not intended to limit the scope of the present disclosure. In some embodiments, fewer drawings, additional drawings, combined drawings, or subdivided drawings may be used. In some embodiments, features illustrated in one figure may be used in combination with features illustrated in one or more other figures.

### DETAILED DESCRIPTION OF THE INVENTION

The present disclosure provides methods, systems, and non-transitory computer-readable media for deriving characteristic physical parameters associated with deoxyribonucleic acid (DNA) from Recognition Physics principles without requiring empirically fitted DNA-specific parameters as inputs to the core derivation. In preferred embodiments, the disclosed framework operates as a first-principles derivation architecture that receives a target DNA parameter, selects a derivation pathway appropriate to the target parameter, performs one or more computations using universal physical constants, forced Recognition Science constants, and defined structural relationships, and outputs a numerical result in physical units together with one or more associated derivation records, intermediate values, validation records, or downstream-use records.

In preferred embodiments, the present disclosure is directed to derivation of a linked family of characteristic DNA parameters rather than generation of unrelated isolated values. In such embodiments, one derived quantity can serve as a base quantity, scaling quantity, dependency input, or consistency anchor for another derived quantity. By way of example and not limitation, a characteristic DNA spatial parameter can support derivation of a helical pitch parameter, a coherence-energy parameter can support derivation of a base-pair stabilization parameter, and one or more structural and energetic parameters can support derivation of a characteristic activity or rate parameter. This linked framework improves internal consistency and supports coherent downstream use in modeling, design, validation, and engineering workflows.

As used herein, the term “characteristic DNA parameter” refers to a physical, geometric, energetic, kinetic, structural, multiplicity-dependent, or derived quantity associated with DNA or a DNA-related system. A characteristic DNA parameter can be a direct output of a derivation pathway or can be derived from one or more previously derived values. Characteristic DNA parameters can include, by way of example and not limitation, a characteristic DNA length parameter, a helical pitch parameter, a coherence-energy parameter, a base-pair stabilization parameter, a rate parameter, a groove-related parameter, a rigidity parameter, a coupling parameter, a density parameter, a normalized factor, a coherence-level parameter, a sequence-dependent parameter, or an analog-specific parameter.

As used herein, the term “parameter-free” means that the core derivation of the target parameter does not require empirically fitted DNA-specific coefficients or empirically tuned DNA-specific constants as inputs to generate the target value. In preferred embodiments, parameter-free derivation can nonetheless use one or more universal physical constants, one or more forced Recognition Science constants, one or more discrete structural multiplicities, one or more target-parameter identifiers, one or more analog or system descriptors, and one or more rule-based or theorem-based selection relations. In further embodiments, experimentally observed values, literature values, benchmark values, or measured values may be used after the core derivation for comparison, validation, ranking, reporting, confidence generation, or acceptance testing, without altering the parameter-free character of the core derivation itself.

For avoidance of doubt, a discrete structural count, a sequence composition count, a selected analog identifier, a target-parameter identifier, a pathway-selection value, or a post-derivation validation reference is not an empirically fitted DNA-specific coefficient merely because it is used in connection with DNA. In preferred embodiments, the excluded inputs are empirically tuned coefficients used to fit the disclosed derivation equations to measured DNA parameter values.

As used herein, the term “Recognition Physics” and the term “Recognition Science” refer to the foundational framework from which one or more forced constants, cost relations, scaling relations, and derivation rules are obtained. In preferred embodiments relevant to the present disclosure, the Recognition Physics framework provides a characteristic optimization constant X_opt and one or more scale cascades from which DNA-relevant outputs can be derived.

In preferred embodiments, a characteristic optimization constant is given by X_opt=phi/pi, where phi=(1+sqrt(5))/2. In further preferred embodiments, a cost architecture is expressed by J(x)=0.5*(x+1/x)−1. In still further preferred embodiments, scale cascades are defined relative to Planck-scale anchor quantities. By way of example and not limitation, a length cascade can be expressed as r_n=L_Planck*(X_opt)\hat{ }n, and an energy cascade can be expressed as E_n=E_Planck*(X_opt)\hat{ }n, where n is a selected index, L_Planck is Planck length, and E_Planck is Planck energy.

In preferred embodiments, the derivation architecture uses one or more pathway classes selected according to the type of target parameter. In some embodiments, the pathway classes include cascade indexing, geometric scaling, and efficiency modulation. In additional embodiments, one or more related pathways, hybrid pathways, dependent pathways, or rule-based composite pathways may be used. The use of pathway classes allows the derivation system to treat different categories of target parameters in a unified but not rigid manner.

In a cascade indexing embodiment, a target parameter is derived from a Planck-scale anchor quantity by selecting an index n and applying X_opt scaling. In some embodiments, a length-type parameter is derived according to a relation of the form P=L_Planck*(X_opt)\hat{ }n. In other embodiments, an energy-type parameter is derived according to a relation of the form P=E_Planck*(X_opt)\hat{ }n. In still other embodiments, another anchor quantity and another cascade relation may be used if appropriate to the parameter class under consideration.

In a geometric scaling embodiment, a target parameter is derived from one or more previously derived parameters using one or more geometry-related scale factors, closure relations, self-similarity relations, or structural scale relations. In some embodiments, a geometry-related factor is expressed in terms of phi. In other embodiments, a geometric scaling relation can include an integer exponent, a rational exponent, a closure-based multiplicative factor, or another defined structural factor.

In an efficiency modulation embodiment, a target parameter is derived from a source quantity by applying an X_opt factor, a multiplicity factor, a sequence factor, a demodulation factor, a structural count, or another discrete factor. In some embodiments, the modulation factor includes a hydrogen-bond multiplicity. In other embodiments, the modulation factor includes a rate-efficiency term, a coherence-scaling term, a geometric occupancy factor, or another rule-based factor.

In preferred embodiments, the present disclosure provides a canonical five-parameter DNA framework. The canonical framework includes a characteristic DNA length parameter X_DNA, a characteristic helical pitch parameter P0, a characteristic coherence-energy parameter E_coh, a characteristic base-pair stabilization parameter E_bp, and a characteristic rate parameter R0. In preferred embodiments, these five parameters are sufficient to define a foundational DNA parameter set from which one or more extended parameters, consistency relations, design constraints, or downstream engineering values can be generated.

In preferred embodiments, the characteristic DNA length parameter X_DNA is derived according to X_DNA=L_Planck*(X_opt)\hat{ }(−90). In some embodiments, X_DNA represents a characteristic DNA spatial scale associated with inter-strand recognition, groove-related geometry, or another structural distance scale. In preferred embodiments, X_DNA is used as a base quantity for one or more downstream geometric derivations, including derivation of helical pitch and characteristic bases-per-turn values.

In preferred embodiments, the characteristic helical pitch parameter P0 is derived according to P0=X_DNA*(phi\hat{ }2). In some embodiments, P0 corresponds to a characteristic axial pitch of a DNA helix. In further embodiments, the quantity P0/(X_DNA/4) provides a characteristic bases-per-turn value. In preferred embodiments, P0 is a dependent geometric output derived from the previously derived X_DNA quantity together with a geometry-related scaling relation.

In preferred embodiments, the characteristic coherence-energy parameter E_coh is derived according to E_coh=E_Planck*(X_opt)\hat{ }(101). In some embodiments, E_coh corresponds to a canonical coherence-associated energy scale for DNA. In further embodiments, E_coh can be used directly or can serve as a base quantity for one or more scaled coherence values, base-pair stabilization values, design thresholds, or validation conditions.

In preferred embodiments, the characteristic base-pair stabilization parameter E_bp is derived according to E_bp=E_coh*X_opt*N_H, where N_H is a hydrogen-bond multiplicity parameter. In some embodiments, N_H is a default multiplicity value representing an average base-pair multiplicity. In other embodiments, N_H is sequence-specific, region-specific, base-pair-specific, library-specific, or analog-specific. In preferred embodiments, E_bp is derived from the previously derived coherence-energy quantity E_coh together with an X_opt factor and a structural multiplicity factor.

In preferred embodiments, the characteristic rate parameter R0 is derived according to R0=(E_coh/h)*(P0/(X_DNA/4))*(2/(phi\hat{ }62)), where h is Planck's constant. In some embodiments, R0 represents a characteristic DNA-related rate scale. In preferred embodiments, R0 is derived from the previously derived coherence-energy and geometric quantities and a biological efficiency factor. In some embodiments, R0 is expressed in bases per second or another suitable unit.

In preferred embodiments, each of the canonical five parameters is positive. In some embodiments, one or more ordering relations among the canonical parameters are also satisfied. By way of example and not limitation, P0 can exceed X_DNA due to the geometry-related factor phi\hat{ }2, and E_bp can exceed E_coh when the product X_opt*N_H is greater than one. In preferred embodiments, the derivation system can generate one or more positivity records, ordering records, or consistency records together with the primary numerical outputs.

In some embodiments, the quantity N_bp_per_turn is derived according to N_bp_per_turn=P0/(X_DNA/4). In preferred embodiments, substitution of the geometric pitch relation yields N_bp_per_turn=4*(phi\hat{ }2). In some embodiments, the bases-per-turn quantity is used in derivation of the characteristic rate parameter R0. In other embodiments, the bases-per-turn quantity is separately reported, validated, compared, or used as a design or screening constraint.

In some embodiments, one or more additional parameters are derived from the canonical five outputs. Such extended parameters can include, by way of example and not limitation, a groove-related ratio, a coupling quantity, a rigidity quantity, a density-like quantity, a normalized factor, a stability quantity, a scaled coherence quantity, a scaled rate quantity, or another dependent quantity. In preferred embodiments, the derivation system can derive a selected subset or all of the canonical parameters and one or more extended parameters in a single execution session.

In some embodiments, a scaled coherence parameter is derived according to E_n=n*E_coh, where n is an integer, a selected coherence level, or another permitted scaling factor. In other embodiments, a scaled rate quantity is derived according to R=n*R0 or according to another scaling relation based on R0. In preferred embodiments, such scaled values are used in downstream design, screening, construct selection, coherence-level evaluation, or practical implementation workflows.

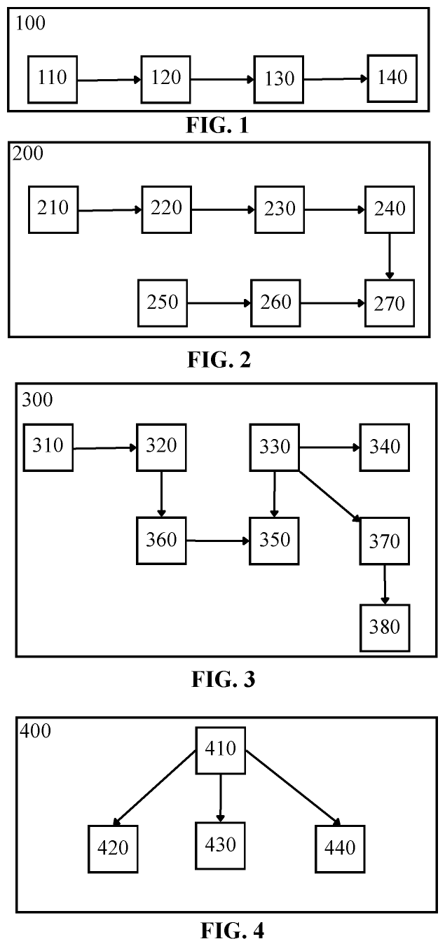

FIG. 1 illustrates an example derivation method 100 for deriving one or more characteristic DNA parameters from Recognition Physics principles. In the illustrated embodiment, method 100 includes a target-parameter identification step 110, a pathway-selection step 120, a derivation step 130, and an output-generation step 140. In some embodiments, one or more optional validation, storage, export, or downstream-use steps may also be included.

In the target-parameter identification step 110, the system receives or determines a target parameter to be derived. In some embodiments, the target parameter is one of X_DNA, P0, E_coh, E_bp, or R0. In other embodiments, the target parameter is an extended parameter, a sequence-specific parameter, an analog-specific parameter, or a parameter family. In some embodiments, the target parameter is received from a user interface, a script, an application programming interface, a batch process, a design pipeline, or a validation workflow.

In the pathway-selection step 120, the system classifies the target parameter and selects a derivation pathway appropriate to the target parameter class. In preferred embodiments, the pathway-selection step 120 assigns the target parameter to one of cascade indexing, geometric scaling, or efficiency modulation. In some embodiments, the selection additionally depends on whether the target parameter is a base quantity, a dependent quantity, a sequence-dependent quantity, or an analog-modified quantity.

In the derivation step 130, the system performs one or more calculations using one or more permitted constants and one or more permitted structural relations. In some embodiments, the derivation step 130 includes selecting a cascade index, applying an X_opt scaling relation, applying a geometry-related factor, applying a multiplicity factor, computing one or more intermediate quantities, and generating a final numerical value. In preferred embodiments, the derivation step 130 does not require empirically fitted DNA-specific coefficients.

In the output-generation step 140, the system outputs the derived value in one or more desired units. In some embodiments, the output-generation step 140 additionally outputs a symbolic equation, one or more intermediate expressions, one or more intermediate values, a derivation trace, a consistency check, a validation comparison, a stored parameter record, or a downstream-use record. In some embodiments, the output is displayed locally. In other embodiments, the output is transmitted to another software system, stored in a database, or returned through a network service.

FIG. 2 illustrates an example derivation system 200 configured to generate characteristic DNA parameters. In the illustrated embodiment, derivation system 200 includes a target parameter identifier 210, a pathway selector 220, a cascade-index selector 230, a calculator 240, a derivation trace generator 250, a validation module 260, and a storage or reporting module 270. In some embodiments, derivation system 200 also includes one or more user interfaces, network interfaces, unit-conversion modules, export modules, security modules, or workflow-integration modules.

In some embodiments, target parameter identifier 210 receives or determines an identifier corresponding to a target parameter. In some embodiments, the identifier specifies a single canonical parameter. In other embodiments, the identifier specifies a multi-parameter run, a parameter family, a sequence-specific output, an analog class, or a downstream design request. In some embodiments, target parameter identifier 210 also receives metadata including a unit preference, precision setting, validation option, analog type, sequence descriptor, or output format preference.

In some embodiments, pathway selector 220 classifies the requested parameter and selects an appropriate derivation pathway. In preferred embodiments, pathway selector 220 selects among cascade indexing, geometric scaling, and efficiency modulation. In other embodiments, pathway selector 220 may invoke a dependent pathway, hybrid pathway, rule-table pathway, or stored derivation template. In some embodiments, pathway selector 220 communicates with cascade-index selector 230 when the selected pathway requires determination of one or more cascade indices or related structural selection quantities.

In some embodiments, cascade-index selector 230 determines one or more index values, exponents, multiplicity values, or structural selection quantities used in the derivation. In preferred embodiments, cascade-index selector 230 operates according to a Minimal Overhead framework or another Recognition Physics rule-based architecture. In some embodiments, cascade-index selector 230 uses a theorem-based relation. In other embodiments, cascade-index selector 230 uses a rule table, a lookup map, an integer-search routine, an optimization routine, a certificate-driven solver, or a hybrid thereof.

In some embodiments, calculator 240 performs the actual numerical or symbolic computation of the target parameter. In some embodiments, calculator 240 operates on exact symbolic expressions. In other embodiments, calculator 240 operates on floating-point values, arbitrary-precision values, interval-bounded values, or combinations thereof. In preferred embodiments, calculator 240 computes one or more canonical parameters and optionally computes one or more intermediate, dependent, extended, or validation-related quantities.

In some embodiments, derivation trace generator 250 generates a structured derivation record associated with a derived output. In some embodiments, the trace includes the target parameter name, pathway class, selected constants, selected indices, selected factors, intermediate equations, intermediate values, unit conversions, final result, and one or more optional proof references or comparison references. In preferred embodiments, derivation trace generator 250 outputs a machine-readable record that can be exported, stored, transmitted, or displayed.

In some embodiments, validation module 260 compares one or more derived outputs with one or more reference values, benchmark values, internally linked consistency relations, or downstream acceptance conditions. In some embodiments, validation module 260 computes a percent difference, error range, ranking score, confidence indication, pass/fail flag, or internal consistency result. In preferred embodiments, validation module 260 operates after the core derivation and does not change the parameter-free character of the core derivation itself.

In some embodiments, storage or reporting module 270 stores or reports one or more derived parameter records. In some embodiments, storage or reporting module 270 stores a parameter value, a derivation trace, a comparison table, an exportable report, or a downstream workflow record in a local database, remote database, file system, cloud repository, versioned record store, or external reporting service. In some embodiments, storage or reporting module 270 also provides one or more application programming interface responses or user-facing reports.

FIG. 3 illustrates an example dependency architecture 300 among canonical derived DNA parameters. In the illustrated embodiment, dependency architecture 300 includes a characteristic DNA length parameter node 310 corresponding to X_DNA, a helical pitch parameter node 320 corresponding to P0, a coherence-energy parameter node 330 corresponding to E_coh, a base-pair stabilization parameter node 340 corresponding to E_bp, and a characteristic rate parameter node 350 corresponding to R0. In some embodiments, one or more additional dependent nodes may also be included, including a bases-per-turn node, a scaled coherence node, a scaled rate node, or one or more extended parameter nodes.

In preferred embodiments, dependency architecture 300 reflects that X_DNA at node 310 serves as a base spatial quantity from which P0 at node 320 is derived. In preferred embodiments, E_coh at node 330 serves as a base energetic quantity from which E_bp at node 340 is derived. In preferred embodiments, R0 at node 350 depends on both structural and energetic quantities, including P0 at node 320, X_DNA at node 310, and E_coh at node 330. This linked dependency architecture supports internal consistency and facilitates downstream design and validation use.

In some embodiments, dependency architecture 300 further includes a bases-per-turn relation 360 derived from P0 and X_DNA. In preferred embodiments, bases-per-turn relation 360 is given by N_bp_per_turn=P0/(X_DNA/4). In some embodiments, dependency architecture 300 also includes a scaled coherence relation 370 given by E_n=n*E_coh and a scaled rate relation 380 given by R=n*R0 or another scaling relation derived from R0. Such extended relations can be used in downstream design, screening, coherence-level evaluation, or construct-selection workflows.

FIG. 4 illustrates an example pathway-selection architecture 400. In the illustrated embodiment, pathway-selection architecture 400 includes a target classification stage 410, a cascade-indexing branch 420, a geometric-scaling branch 430, and an efficiency-modulation branch 440. In some embodiments, pathway-selection architecture 400 also includes one or more dependent-pathway, hybrid-pathway, analog-pathway, or validation-pathway branches.

In some embodiments, target classification stage 410 classifies a target parameter according to one or more of physical dimension, dependency role, parameter type, analog type, structural class, or desired output family. In preferred embodiments, a length-type or energy-type base quantity is directed toward cascade-indexing branch 420, a dependent structural quantity is directed toward geometric-scaling branch 430, and a multiplicity-dependent or efficiency-weighted quantity is directed toward efficiency-modulation branch 440.

In some embodiments, cascade-indexing branch 420 applies a selected index to a Planck-scale anchor quantity using X_opt scaling. In some embodiments, geometric-scaling branch 430 applies a geometry-related scale factor such as phi\hat{ }2 or another closure-related factor to a previously derived parameter. In some embodiments, efficiency-modulation branch 440 applies X_opt together with a multiplicity, sequence, or structural factor to a previously derived source quantity. In preferred embodiments, each branch produces both a numerical output and a derivation trace.

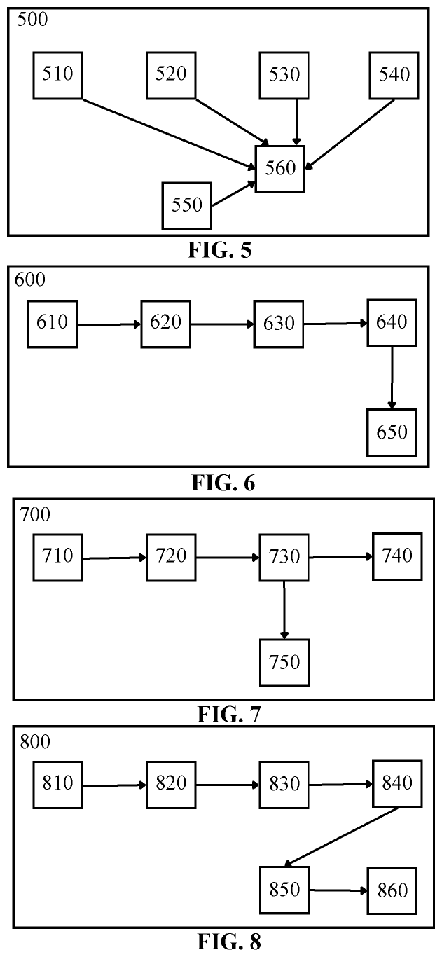

FIG. 5 illustrates an example Minimal Overhead selection framework 500 for determining one or more cascade indices, exponents, multiplicity values, or structural factors used in derivation of one or more characteristic DNA parameters. In the illustrated embodiment, Minimal Overhead selection framework 500 includes a target-identity input 510, a structural-class input 520, a dimensionality input 530, a multiplicity input 540, a stability or interaction input 550, and a selection output 560. In some embodiments, one or more additional inputs may be used, including an analog-type input, a dependency-role input, a scale-class input, or a theorem-certificate input.

In some embodiments, target-identity input 510 identifies which target parameter is being derived. In some embodiments, structural-class input 520 identifies whether the target is associated with spatial structure, helical geometry, coherence-associated energy, multiplicity-weighted stabilization, or rate-related activity. In some embodiments, dimensionality input 530 characterizes the target system as one-dimensional, quasi-one-dimensional, dual-recognition, or another recognized physical class. In some embodiments, multiplicity input 540 identifies a discrete structural multiplicity such as hydrogen-bond count or another count-based quantity. In some embodiments, stability or interaction input 550 identifies a relevant interaction type, stability condition, or analog constraint.

In preferred embodiments, selection output 560 provides one or more selected values used in the derivation. In some embodiments, selection output 560 includes a cascade index n. In other embodiments, selection output 560 includes a geometry-related exponent, a multiplicity factor, a closure factor, a demodulation factor, or another rule-based quantity. In preferred embodiments, the selected values are determined according to a Recognition Physics relation or rule-based architecture rather than by empirical fitting to DNA-specific datasets.

In one preferred embodiment, Minimal Overhead selection framework 500 outputs a cascade index of −90 for derivation of X_DNA. In one preferred embodiment, Minimal Overhead selection framework 500 outputs a cascade index of 101 for derivation of E_coh. In some embodiments, equivalent theorem-based, rule-table, or certificate-driven implementations can be used to obtain the same selections. In some embodiments, different analog types, structural classes, or target categories may yield different selected values while preserving the overall architecture.

In some embodiments, the rule-based selection architecture is implemented as a finite stored rule table in memory, the rule table comprising a plurality of entries each keyed by (i) a target-parameter identifier or target-parameter type, (ii) a structural class, and optionally (iii) a dimensionality class and/or (iv) a multiplicity/stability class, and each entry mapping the key to one or more permitted pathway-selection values. In such embodiments, the system determines a canonical key for the requested target parameter, queries the rule table using the canonical key and associated classification values, retrieves the one or more permitted pathway-selection values, and selects a pathway-selection value from the retrieved set according to at least one Recognition Physics consistency rule. In one example, for a length-type base parameter associated with a DNA minor-groove characteristic length, the retrieved permitted set includes n=−90; and for a coherence-energy base parameter, the retrieved permitted set includes n=101. In some embodiments, when no rule-table entry exists for the canonical key, the system outputs an error flag and declines to derive the requested parameter.

In some embodiments, selection output 560 is generated according to a rule-based selector that operates on a finite set of candidate pathway-selection values. In such embodiments, the selector receives as inputs at least target-identity input 510, structural-class input 520, dimensionality input 530, multiplicity input 540, and stability or interaction input 550, and evaluates a finite candidate set of pathway-selection values against one or more Recognition Physics consistency criteria.

In one example embodiment, the selector operates according to the following procedure: (i) determine whether the target parameter is a base parameter or a dependent parameter; (ii) if the target parameter is a base parameter, determine whether the target parameter is a length-type base parameter or an energy-type base parameter; (iii) generate a candidate set of cascade indices n for the identified parameter type; (iv) evaluate the candidate set against at least one rule reflecting dimensionality, recognition multiplicity, structural class, or stability condition; (v) select the cascade index that satisfies the rule-based Recognition Physics selection architecture for the target parameter class; and (vi) output the selected cascade index as selection output 560.

In one preferred embodiment, where the target parameter is X_DNA, the selector classifies the target as a DNA spatial base parameter associated with a one-dimensional or quasi-one-dimensional dual-recognition structure and outputs n=−90. In one preferred embodiment, where the target parameter is E_coh, the selector classifies the target as a coherence-associated energy base parameter and outputs n=101.

In some embodiments, if the target parameter is a dependent geometric parameter, the selector does not output a new cascade index and instead outputs a geometry-related factor or closure factor. In one preferred embodiment, where the target parameter is P0, the selector outputs a geometry-related factor equal to phi\hat{ }2 for application to X_DNA. In some embodiments, if the target parameter is a multiplicity-dependent parameter, the selector outputs a multiplicity factor based on a structural count. In one preferred embodiment, where the target parameter is E_bp, the selector outputs N_H as a hydrogen-bond multiplicity factor.

In some embodiments, the selector is implemented as a theorem-backed lookup structure, a rule table, an integer-search routine, an optimization routine constrained by Recognition Physics rules, or a hybrid thereof. In preferred embodiments, the selector does not determine the pathway-selection value by regression fitting to measured DNA-specific parameter values.

FIG. 6 illustrates an example X_DNA derivation pathway 600. In the illustrated embodiment, X_DNA derivation pathway 600 includes a Planck-length anchor 610, an optimization constant input 620, a selected index input 630, a scaling operation 640, and an X_DNA output 650. In preferred embodiments, Planck-length anchor 610 corresponds to L_Planck, optimization constant input 620 corresponds to X_opt, selected index input 630 corresponds to −90, scaling operation 640 applies the length cascade relation, and X_DNA output 650 corresponds to the resulting characteristic DNA length parameter.

In preferred embodiments, X_DNA derivation pathway 600 computes X_DNA according to X_DNA=L_Planck*(X_opt)\hat{ }(−90). In some embodiments, X_DNA output 650 is reported in meters, Angstroms, or both. In some embodiments, X_DNA output 650 is interpreted as a characteristic DNA distance scale associated with inter-strand recognition, groove-related geometry, or another structural spatial quantity. In preferred embodiments, X_DNA output 650 is stored as a canonical base parameter for use in one or more downstream derivations.

In some embodiments, X_DNA derivation pathway 600 additionally generates one or more validation or reporting records. Such records can include a symbolic expression record, a numerical precision record, a unit-conversion record, a consistency record, a comparison-to-reference record, or a downstream dependency tag. In some embodiments, X_DNA output 650 is then supplied to the P0 derivation pathway described below.

FIG. 7 illustrates an example P0 derivation pathway 700. In the illustrated embodiment, P0 derivation pathway 700 includes an X_DNA input 710, a geometry factor input 720, a scaling operation 730, a P0 output 740, and an optional bases-per-turn output 750. In preferred embodiments, X_DNA input 710 corresponds to the previously derived X_DNA value, geometry factor input 720 corresponds to phi\hat{ }2, scaling operation 730 multiplies X_DNA by phi\hat{ }2, P0 output 740 corresponds to the resulting helical pitch parameter, and optional bases-per-turn output 750 corresponds to P0/(X_DNA/4).

In preferred embodiments, P0 derivation pathway 700 computes P0 according to P0=X_DNA*(phi\hat{ }2). In some embodiments, P0 output 740 is interpreted as a characteristic helical pitch parameter for DNA. In some embodiments, optional bases-per-turn output 750 is computed according to N_bp_per_turn=P0/(X_DNA/4). In preferred embodiments, substitution yields N_bp_per_turn=4*(phi\hat{ }2). In some embodiments, optional bases-per-turn output 750 is used in the R0 derivation pathway described below.

In some embodiments, P0 derivation pathway 700 generates one or more additional outputs or records, including a positivity record, an ordering record showing that P0 exceeds X_DNA, a validation record, a downstream geometry tag, or a derived-value export record. In some embodiments, P0 output 740 is stored as a canonical geometric parameter for one or more later workflows.

FIG. 8 illustrates an example E_coh derivation pathway 800. In the illustrated embodiment, E_coh derivation pathway 800 includes a Planck-energy anchor 810, an optimization constant input 820, a selected index input 830, a scaling operation 840, and an E_coh output 850. In preferred embodiments, Planck-energy anchor 810 corresponds to E_Planck, optimization constant input 820 corresponds to X_opt, selected index input 830 corresponds to 101, scaling operation 840 applies the energy cascade relation, and E_coh output 850 corresponds to the resulting characteristic coherence-energy parameter.

In preferred embodiments, E_coh derivation pathway 800 computes E_coh according to E_coh=E_Planck*(X_opt)\hat{ }(101). In some embodiments, E_coh output 850 is reported in electronvolts, joules, or both. In some embodiments, E_coh output 850 is interpreted as a canonical coherence-associated energy scale for DNA. In preferred embodiments, E_coh output 850 is stored as a canonical energetic parameter for downstream use in base-pair stabilization, scaled coherence, stability evaluation, design screening, or construct selection.

In some embodiments, E_coh derivation pathway 800 additionally generates one or more scaled coherence values. In some embodiments, a scaled coherence output 860 is generated according to E_n=n*E_coh, where n is an integer or other permitted scaling factor. In some embodiments, scaled coherence output 860 is used in downstream coherence-level design, construct screening, or acceptance testing.

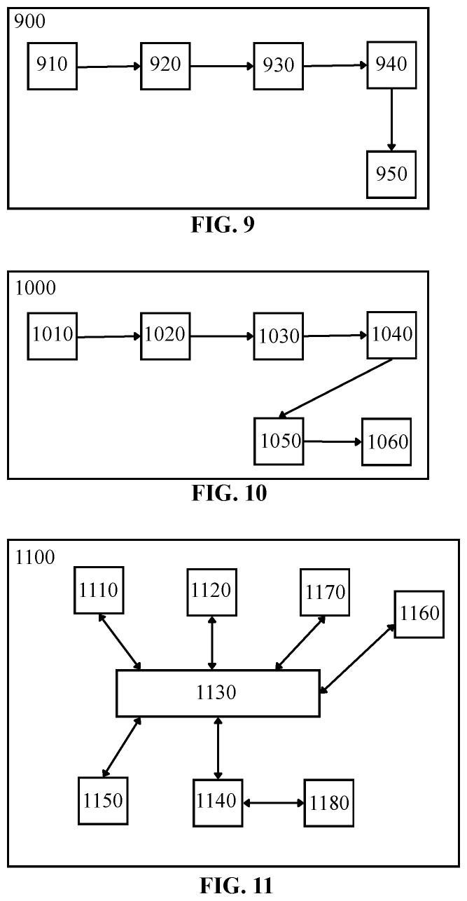

FIG. 9 illustrates an example E_bp derivation pathway 900. In the illustrated embodiment, E_bp derivation pathway 900 includes an E_coh input 910, an X_opt input 920, a multiplicity input 930, a modulation operation 940, and an E_bp output 950. In some embodiments, multiplicity input 930 corresponds to a hydrogen-bond multiplicity parameter N_H. In preferred embodiments, modulation operation 940 applies the relation E_bp=E_coh*X_opt*N_H.

In preferred embodiments, E_bp derivation pathway 900 computes E_bp according to E_bp=E_coh*X_opt*N_H. In some embodiments, multiplicity input 930 is a default average value. In other embodiments, multiplicity input 930 is sequence-specific, region-specific, local-window-specific, base-pair-specific, or analog-specific. In preferred embodiments, E_bp output 950 is reported in electronvolts, joules, or energy-per-mole units.

In one preferred sequence-specific embodiment, multiplicity input 930 is determined according to N_H=(2*#AT+3*#CG)/(#AT+#CG), where #AT represents the number of adenine-thymine base pairs and #CG represents the number of cytosine-guanine base pairs in a selected sequence, region, library, or local window. In some embodiments, a purely adenine-thymine region can be assigned N_H=2, and a purely cytosine-guanine region can be assigned N_H=3. In other embodiments, modified bond structures or analog-specific bond counts may be used.

In some embodiments, E_bp derivation pathway 900 generates one or more additional records including a sequence-composition record, a local-window record, a validation record, a comparison-to-reference record, or a downstream stabilization tag. In preferred embodiments, E_bp output 950 is stored as a canonical stabilization parameter or as a sequence-specific stabilization value for later design, screening, or reporting use.

FIG. 10 illustrates an example R0 derivation pathway 1000. In the illustrated embodiment, R0 derivation pathway 1000 includes an E_coh input 1010, a bases-per-turn input 1020, an efficiency-factor input 1030, a rate computation operation 1040, and an R0 output 1050. In preferred embodiments, E_coh input 1010 corresponds to the canonical coherence-energy value, bases-per-turn input 1020 corresponds to P0/(X_DNA/4), efficiency-factor input 1030 corresponds to 2/(phi\hat{ }62), rate computation operation 1040 applies the disclosed rate relation, and R0 output 1050 corresponds to the resulting characteristic rate parameter.

In preferred embodiments, R0 derivation pathway 1000 computes R0 according to R0=(E_coh/h)*(P0/(X_DNA/4))*(2/(phi\hat{ }62)). In some embodiments, R0 output 1050 is reported in bases per second or another suitable rate unit. In some embodiments, R0 output 1050 is stored as a canonical activity or rate parameter. In some embodiments, a scaled rate output 1060 is additionally generated according to R=n*R0 or another scaling relation derived from R0.

In some embodiments, R0 derivation pathway 1000 generates one or more downstream-use records indicating that the derived rate value can be used for screening, design ranking, throughput estimation, activity evaluation, or technical reporting. In some embodiments, R0 output 1050 is also subjected to one or more consistency checks using the previously derived X_DNA, P0, and E_coh values.

In preferred embodiments, the disclosed derivation system derives two or more of the canonical five parameters during a single computational session. In preferred embodiments, all five canonical parameters are derived in a single execution and stored as a linked parameter family. In some embodiments, the linked parameter family includes dependency metadata indicating that X_DNA supports derivation of P0, that E_coh supports derivation of E_bp, and that X_DNA, P0, and E_coh support derivation of R0.

In some embodiments, the system generates a derivation trace for each output parameter. A derivation trace can include, by way of example and not limitation, the target parameter identifier, pathway class, selected constants, selected indices, selected factors, intermediate equations, intermediate values, unit conversions, final result, and one or more optional validation references. In preferred embodiments, the derivation trace is machine-readable and can be exported, stored, transmitted, displayed, or incorporated into a downstream technical workflow.

In some embodiments, the system generates one or more consistency checks across the derived parameter family. By way of example and not limitation, the system can verify that P0 exceeds X_DNA, that E_bp exceeds E_coh for a selected multiplicity regime, that bases-per-turn equals P0 divided by X_DNA/4, and that R0 is computed from previously derived structural and energetic values according to the disclosed relation. In some embodiments, a failed consistency check can trigger a diagnostic flag, alternate-pathway report, validation warning, or recomputation routine.

In some embodiments, the present disclosure supports sequence-specific outputs without requiring empirically fitted DNA-specific coefficients. In such embodiments, one or more discrete structural quantities associated with the sequence can be used as permitted inputs to the core derivation or to a dependent derivation stage. Examples include hydrogen-bond multiplicity, local composition class, motif class, mismatch count, base-pair-type distribution, or another non-fitted structural count. In preferred embodiments, such quantities are used as rule-based multiplicity inputs rather than regression-fit coefficients.

In some embodiments, a sequence-specific embodiment operates on a full DNA sequence. In other embodiments, the sequence-specific embodiment operates on a selected region, local window, motif instance, consensus region, candidate library, or pooled sequence family. In still further embodiments, global and local parameter values can both be derived and reported, such as an overall average stabilization quantity and one or more local stabilization values for sliding windows or functional regions.

In some embodiments, the present disclosure is applied not only to natural DNA but also to one or more nucleic-acid analogs or related systems. Such systems can include, by way of example and not limitation, ribonucleic acid, peptide nucleic acid, xDNA systems, Hachimoji systems, modified base systems, modified backbone systems, hybrid nucleic-acid systems, nucleic-acid-protein hybrid systems, or engineered constructs that preserve a recognizable nucleic-acid-like architecture.

In some embodiments, an analog-specific derivation differs from a DNA derivation by modifying one or more rule-based inputs or selection rules while preserving the same foundational derivation architecture. By way of example and not limitation, an analog-specific embodiment can modify one or more of dimensionality, recognition multiplicity, structural class, geometric scaling factor, closure relation, stability criterion, sequence multiplicity rule, or cascade-index selection rule. In this way, the framework can preserve a common first-principles structure while adapting to different molecular architectures.

In some embodiments, an RNA embodiment uses one or more altered geometric or selection rules reflecting a different helical architecture from canonical B-form DNA. In some embodiments, a PNA embodiment uses one or more altered multiplicity or structural descriptors reflecting a different backbone architecture. In further embodiments, xDNA, Hachimoji, or other engineered systems can be treated using analogous modified rule sets without departing from the disclosed framework.

In some embodiments, the derived parameters are used in one or more downstream engineering workflows. Such workflows can include DNA construct design, coherence-first design, simulator initialization, sequence screening, parameter library generation, structural validation, synthesis selection, experimental planning, or network-based reporting. In preferred embodiments, the present disclosure therefore functions not merely as a mathematical derivation procedure but as a practical parameter-generation architecture for technical use.

In some embodiments, the derived parameters are used to populate or constrain a formal DNA design representation. In some embodiments, the design representation includes sequence, geometry, and coherence-associated quantities. In preferred embodiments, one or more of X_DNA, P0, E_coh, E_bp, R0, E_n, or a related dependent quantity is used to define, constrain, validate, or score a candidate design or candidate construct.

In some embodiments, a coherence-first design embodiment begins with selection of a target coherence level and derives one or more downstream quantities used to define an acceptable construct space. In some embodiments, a scaled coherence quantity E_n=n*E_coh is used together with one or more structural or stability conditions. In some embodiments, a scaled rate quantity based on R0 is also used. In further embodiments, candidate constructs or candidate sequence sets can be screened for compatibility with one or more derived energy, geometry, stability, or rate conditions.

In some embodiments, the derived numerical value is not merely displayed, but is used to configure a downstream technical process operating on a DNA sequence, a candidate DNA construct, a candidate nucleic-acid analog, or a validation workflow. In some embodiments, the downstream technical process includes automated acceptance or rejection of a candidate construct based on whether the candidate construct satisfies one or more thresholds derived from X_DNA, P0, E_coh, E_bp, R0, E_n, or R.

In some embodiments, a simulator initialization workflow uses one or more of X_DNA, P0, E_coh, E_bp, or R0 as initialization parameters, constraint parameters, or validation parameters for a structural, energetic, coherence-related, or kinetic model of a DNA system. In some embodiments, the simulator is automatically configured by the derivation system 200 using the derived numerical value and the derivation trace.

In some embodiments, a construct-selection workflow receives a plurality of candidate DNA constructs and automatically ranks or filters the candidate DNA constructs based on compatibility with one or more derived quantities. In some embodiments, the compatibility evaluation includes comparing a candidate geometry against X_DNA or P0, comparing a candidate stabilization profile against E_bp, comparing a candidate coherence level against E_coh or E_n, or comparing a candidate activity profile against R0 or R.

In some embodiments, a laboratory planning workflow uses one or more disclosed derived parameters to generate a recommended synthesis list, a recommended validation experiment list, or a recommended screening order for candidate constructs. In some embodiments, the workflow is implemented by one or more processors and produces a machine-readable output identifying at least one construct to synthesize, at least one construct to reject, or at least one validation assay to perform.

In some embodiments, the disclosed derivation system is implemented as a software platform executing on one or more processors. In some embodiments, the software platform includes target parameter identifier 210, pathway selector 220, cascade-index selector 230, calculator 240, derivation trace generator 250, validation module 260, and storage or reporting module 270. In some embodiments, the modules are executed on a local workstation. In other embodiments, one or more modules are executed in a cloud environment, distributed environment, server environment, embedded environment, or hybrid environment.

In some embodiments, the disclosed system provides one or more application programming interface services through which an external requester can submit a target parameter request and receive a derived output. In some embodiments, the request identifies the target parameter, analog type, sequence descriptor, output format, precision requirement, or validation option. In some embodiments, the response includes a derived numerical value, equation text, derivation trace, consistency record, and validation record. In some embodiments, the response is encoded in JSON, XML, CSV, text, or another machine-readable format.

In some embodiments, the disclosed system stores one or more parameter records in a database or versioned repository. In some embodiments, a stored parameter record includes a parameter name, pathway class, formula text, constants used, selected indices or factors, output value, units, derivation trace, validation data, and downstream-use tags. In some embodiments, multiple records are stored for multiple sequences, analog types, parameter families, design candidates, or validation states.

Referring to FIG. 11, an example computing system 1100 includes one or more processors 1110, memory 1120, and a bus or interconnect 1130 coupling the components. The computing system 1100 further includes a network interface 1140, an input/output interface 1150, and a data store 1160 storing at least constant libraries and/or parameter records including derived values and derivation traces. In operation, the one or more processors 1110 execute instructions stored in memory 1120 to implement the functions described herein for target parameter identification, pathway selection, pathway-selection value determination, numerical calculation, derivation trace generation, and output reporting.

In some embodiments, computing system 1100 further includes an accelerator or co-processor 1170 coupled to bus/interconnect 1130. In some embodiments, network interface 1140 communicates with at least one external network, remote service, or remote computing resource 1180.

In some embodiments, validation module 260 compares one or more derived values with one or more reference values after derivation. In preferred embodiments, such reference values are not used as inputs to the core derivation. Instead, the values are used for benchmarking, ranking, confidence reporting, acceptance testing, or literature comparison. In some embodiments, validation module 260 outputs a percent-difference table, a ranked comparison table, a pass/fail indication, or a consistency report.

In some embodiments, a validation output generated by validation module 260 includes a machine-readable acceptance flag indicating whether a candidate DNA construct, candidate sequence, or candidate analog satisfies one or more predetermined thresholds based on a derived value selected from X_DNA, P0, E_coh, E_bp, R0, E_n, or R. In some embodiments, the acceptance flag is stored together with the derivation trace in a parameter record or construct-selection record.

In some embodiments, the system generates one or more reports for human or machine consumption. Such reports can include a canonical five-parameter report, a multi-parameter trace report, a sequence-specific stabilization report, an analog-comparison report, a validation report, a design-screening report, or a construct-qualification report. In some embodiments, the report is exported as text, spreadsheet data, a structured document, or a machine-readable API response.

In some embodiments, the disclosed framework is used to support candidate construct selection. In some embodiments, a candidate construct is evaluated using one or more of X_DNA, P0, E_coh, E_bp, R0, E_n, a sequence-specific stabilization value, or a consistency or validation result derived therefrom. In some embodiments, the candidate construct is accepted, rejected, ranked, or modified based at least in part on one or more such quantities.

In some embodiments, the disclosed framework supports synthesis planning or experimental validation planning. In some embodiments, a candidate sequence set or candidate molecular architecture is selected based on one or more derived quantities. In some embodiments, one or more validation experiments are selected based on the predicted structural, energetic, or rate-related properties. In some embodiments, the framework supports automated or semi-automated handoff to a synthesis vendor, an experimental planning system, or a laboratory workflow manager.

In some embodiments, one or more derived parameters are used in connection with functionalized or hybrid systems. Such systems can include DNA-associated chromophores, proteins, nanoparticles, catalysts, donor-acceptor systems, or signaling components. In some embodiments, the derived quantities are used to screen or configure a structural geometry, an energetic compatibility condition, a coherence-level condition, or a functional performance condition for such systems.

In some embodiments, the disclosed framework is embodied in a non-transitory computer-readable medium storing instructions that, when executed by one or more processors, cause the one or more processors to perform one or more of the steps described herein. In some embodiments, the instructions cause the processors to receive a target parameter identifier, select a derivation pathway, perform a parameter-free derivation using one or more permitted constants and structural relations, generate one or more outputs and derivation traces, and optionally store, validate, export, or apply the resulting parameter values.

In some embodiments, the disclosed methods, systems, and media derive a single target parameter, a selected subset of parameters, or a full parameter family for a given DNA system, sequence, analog, or design context. In some embodiments, the derivation order varies depending on the requested outputs. In some embodiments, one or more canonical base parameters are first derived and one or more dependent parameters are then derived therefrom. In other embodiments, a stored canonical parameter is retrieved and used as a source quantity for a dependent derivation.

In some embodiments, the disclosed framework may additionally generate one or more proof-linked or certificate-linked records indicating that a selected cascade relation, index value, or structural relation corresponds to a predetermined Recognition Physics theorem, rule, proof artifact, or certified derivation record. In some embodiments, such proof-linked records are included in a derivation trace or validation report.

The embodiments described herein are illustrative and non-limiting. Features described in connection with one embodiment may be used in combination with features described in connection with another embodiment unless expressly stated otherwise. The disclosed pathway classes, equations, module arrangements, processing orders, and output formats may be varied while preserving the overall inventive concept of deriving characteristic DNA parameters parameter-free from Recognition Physics principles.

Unless the context clearly requires otherwise, singular terms can include plural referents, plural terms can include singular referents, and open-ended terms such as “comprising” are intended to be inclusive and non-exclusive. Numerical ranges, constants, factors, indices, and example implementations described herein may include subranges, endpoint values, equivalent forms, and functionally corresponding implementations consistent with the present disclosure.

Accordingly, the present disclosure provides an integrated first-principles architecture for deriving characteristic DNA parameters, organizing those parameters into a linked family, generating derivation traces and validation outputs, and applying the resulting quantities in practical technical workflows including modeling, design, screening, reporting, construct selection, synthesis planning, and validation.

The following examples are provided to illustrate representative embodiments of the present disclosure and are not intended to limit the scope of the invention. In some embodiments, the equations, values, workflows, and outputs described in the following examples may be modified, reordered, extended, or combined with other disclosed embodiments while remaining within the scope of the present disclosure.

In one example embodiment, the disclosed system derives the canonical five-parameter family for a DNA system during a single execution. In such an embodiment, target parameter identifier 210 receives a multi-parameter request corresponding to X_DNA, P0, E_coh, E_bp, and R0. Pathway selector 220 classifies X_DNA and E_coh as base cascade-derived quantities, classifies P0 as a geometry-dependent quantity, classifies E_bp as a multiplicity-dependent quantity, and classifies R0 as a dependent rate quantity. Cascade-index selector 230 then supplies the selected cascade indices for the applicable base parameters, calculator 240 computes the requested outputs, and derivation trace generator 250 generates a linked trace showing the dependency structure across the full parameter family.

In one preferred example, calculator 240 derives X_DNA according to X_DNA=L_Planck*(X_opt)\hat{ }(−90), where X_opt=phi/pi and phi=(1+sqrt(5))/2. In such an embodiment, calculator 240 may first retrieve or compute X_opt, may retrieve L_Planck from a stored constant library or may compute L_Planck from other physical constants, and may then apply the selected index to generate the X_DNA output. In some embodiments, the result is reported in meters. In some embodiments, the result is additionally converted to Angstroms by unit conversion performed before or after final output generation.

In one preferred example, calculator 240 derives P0 according to P0=X_DNA*(phi\hat{ }2). In such an embodiment, calculator 240 receives the previously derived X_DNA value from X_DNA derivation pathway 600, applies geometry factor input 720 corresponding to phi\hat{ }2, and generates P0 output 740. In some embodiments, optional bases-per-turn output 750 is additionally computed according to N_bp_per_turn=P0/(X_DNA/4). In preferred embodiments, this relation yields N_bp_per_turn=4*(phi\hat{ }2).

In one preferred example, calculator 240 derives E_coh according to E_coh=E_Planck*(X_opt)\hat{ }(101). In such an embodiment, calculator 240 retrieves or computes E_Planck, applies the selected index corresponding to selected index input 830, and generates E_coh output 850. In some embodiments, E_coh output 850 is reported in joules. In other embodiments, E_coh output 850 is converted to electronvolts. In some embodiments, scaled coherence output 860 is additionally generated according to E_n=n*E_coh for one or more selected values of n.

In one preferred example, calculator 240 derives E_bp according to E_bp=E_coh*X_opt*N_H. In a default embodiment, multiplicity input 930 is assigned a default value corresponding to an average base-pair multiplicity. In a sequence-dependent embodiment, multiplicity input 930 is derived from sequence composition. In one preferred sequence-composition embodiment, multiplicity input 930 is computed according to N_H=(2*#AT+3*#CG)/(#AT+#CG). Calculator 240 then generates E_bp output 950 using the selected multiplicity input.

In one preferred example, calculator 240 derives R0 according to R0=(E_coh/h)*(P0/(X_DNA/4))*(2/(phi\hat{ }62)). In such an embodiment, calculator 240 receives E_coh from E_coh derivation pathway 800, receives or computes bases-per-turn input 1020 from the P0 relation, retrieves or computes efficiency-factor input 1030 corresponding to 2/(phi\hat{ }62), and generates R0 output 1050. In some embodiments, scaled rate output 1060 is additionally generated according to R=n*R0 or another scaling relation.

In one example implementation, derivation trace generator 250 outputs a machine-readable record for the canonical five-parameter derivation. In some embodiments, the machine-readable record includes a target-parameter list, pathway-class selections, selected constants, selected indices, multiplicity values, intermediate equations, intermediate values, final values, unit-conversion metadata, and dependency metadata. In some embodiments, the machine-readable record additionally includes a consistency table indicating that P0 exceeds X_DNA, that E_bp exceeds E_coh in a selected multiplicity regime, that bases-per-turn equals P0 divided by X_DNA/4, and that R0 depends on previously derived structural and energetic quantities.

In one sequence-specific example, the disclosed system receives a DNA sequence or sequence descriptor and computes a sequence-specific base-pair stabilization quantity. In such an embodiment, target parameter identifier 210 receives a request for E_bp together with sequence metadata. Pathway selector 220 classifies the request as an efficiency-modulation derivation. Calculator 240 or another associated processing module then determines the number of adenine-thymine base pairs and cytosine-guanine base pairs in the sequence or selected region and computes multiplicity input 930 according to N_H=(2*#AT+3*#CG)/(#AT+#CG). The resulting value is then used to compute E_bp output 950.

In one local-window example, a sequence is divided into multiple windows, and a separate multiplicity input 930 is computed for each window. In such an embodiment, a local stabilization profile is generated across the length of the sequence. In some embodiments, the local stabilization profile is stored as a set of regional E_bp values. In some embodiments, the local stabilization profile is used to identify regions of relatively higher or lower predicted stabilization for subsequent screening, design, or validation use.

In one library-screening example, the system processes a candidate library of DNA sequences and generates one or more derived parameter values for each candidate. In some embodiments, each candidate receives a canonical five-parameter record. In other embodiments, each candidate receives only a selected subset of outputs, such as E_bp, R0, or one or more scaled coherence values. In some embodiments, validation module 260 ranks the candidate sequences according to one or more of stabilization compatibility, coherence-level compatibility, rate compatibility, geometric compatibility, or another downstream technical criterion.

In one analog-specific example, the system receives a request corresponding to a non-DNA nucleic-acid analog. In such an embodiment, target parameter identifier 210 receives metadata identifying the analog type, pathway selector 220 invokes one or more modified selection rules, and cascade-index selector 230 or calculator 240 applies one or more modified structural inputs while preserving the overall first-principles architecture. In some embodiments, the analog is RNA. In some embodiments, the analog is PNA. In some embodiments, the analog is xDNA, Hachimoji, or another engineered nucleic-acid system.

In one RNA example, the system applies one or more modified geometric assumptions reflecting a non-B-form helical architecture. In such an embodiment, the system may preserve one or more cascade-derived energetic or spatial principles while modifying one or more geometry-related scale factors or closure relations. In some embodiments, an RNA-specific pitch-like quantity or rate-like quantity is generated using the modified rule set. In some embodiments, the resulting outputs are stored as analog-specific parameter records distinct from canonical DNA parameter records.

In one PNA example, the system applies one or more modified multiplicity rules, backbone rules, or structural-class rules. In such an embodiment, pathway selector 220 may classify the target parameter under a modified analog pathway, and calculator 240 may compute one or more outputs using altered multiplicity, structural, or stability assumptions suitable for the PNA system. In some embodiments, the resulting values are used to compare candidate PNA designs or to screen for compatibility with one or more downstream engineering criteria.

In one downstream design example, the system uses one or more derived parameters to populate or constrain a formal DNA design representation. In such an embodiment, one or more of X_DNA, P0, E_coh, E_bp, R0, E_n, or a related dependent value is supplied to a design engine or design record. In some embodiments, the design record includes a sequence component, a geometry component, and an energy or coherence component. In some embodiments, the system uses the derived quantities to accept, reject, score, or rank candidate design representations.

In one coherence-first design example, a target coherence level is selected prior to sequence selection. In such an embodiment, scaled coherence output 860 is generated according to E_n=n*E_coh, and one or more derived structural, stability, or rate-related conditions are then applied to define an acceptable design space. In some embodiments, candidate sequences or constructs are screened according to compatibility with the selected coherence level, one or more geometry conditions, one or more stabilization conditions, or one or more activity conditions associated with R0 or a scaled rate value.

In one construct-screening example, the system evaluates a candidate construct using one or more canonical or extended parameters. In such an embodiment, the candidate construct may include a DNA sequence, a geometry definition, one or more attached components, or one or more intended functional uses. In some embodiments, validation module 260 evaluates whether the candidate construct satisfies one or more derived energy thresholds, one or more geometry constraints, one or more rate-related thresholds, or one or more internal consistency relations. In some embodiments, the construct is then ranked, accepted, rejected, or modified based on the evaluation.

In one hybrid-system example, a candidate system includes DNA together with one or more associated chromophores, proteins, nanoparticles, donor-acceptor elements, catalysts, or signaling components. In such an embodiment, one or more derived quantities can be used to evaluate whether the hybrid system satisfies one or more structural compatibility conditions, energetic compatibility conditions, coherence-related conditions, or use-case-specific performance conditions. In some embodiments, the disclosed framework is used to select among multiple candidate hybrid configurations.

In one validation example, validation module 260 compares one or more derived outputs with one or more reference values after core derivation has been completed. In such an embodiment, the reference values may include literature values, experimental values, accepted ranges, internally stored benchmark values, or previously measured values. In some embodiments, validation module 260 computes a difference, percent difference, ranking score, confidence indicator, or pass/fail output. In preferred embodiments, the validation process is separated from the core derivation so that the core derivation remains parameter-free as defined herein.

In one reporting example, storage or reporting module 270 generates a structured report for a derived parameter family. In some embodiments, the report includes canonical values for X_DNA, P0, E_coh, E_bp, and R0, together with one or more symbolic equations, unit conversions, dependency relationships, and validation results. In some embodiments, the report is human-readable. In some embodiments, the report is machine-readable. In some embodiments, the report is formatted as text, structured data, a table, or an application programming interface response.

In one application programming interface embodiment, an external system submits a derivation request through a network interface. In some embodiments, the request identifies a target parameter, target parameter family, sequence, analog type, unit preference, precision preference, validation option, or reporting option. In response, derivation system 200 performs the requested derivation and returns one or more outputs including a numerical value, derivation trace, consistency record, and optional validation result. In some embodiments, the response is encoded in JSON, XML, CSV, text, or another machine-readable format.

In one batch-processing embodiment, a plurality of derivation requests are processed in a single run. In such an embodiment, each request may correspond to a different sequence, target parameter, analog type, or validation state. In some embodiments, the results are stored in a database or versioned repository, allowing later retrieval, comparison, ranking, or export. In some embodiments, a batch-processing embodiment is used to create a parameter library spanning multiple candidate systems or multiple analog classes.

In one non-transitory computer-readable medium embodiment, computer-executable instructions are stored on one or more media and, when executed by one or more processors, cause the processors to perform one or more of the derivation steps described herein. In some embodiments, the instructions cause the processors to receive a target parameter request, classify the parameter, select a derivation pathway, determine one or more pathway-selection values, compute the requested output, generate a derivation trace, and store, export, validate, or transmit the result. In some embodiments, the non-transitory computer-readable medium embodiment supports a single-parameter, multi-parameter, batch, sequence-specific, analog-specific, or design-oriented workflow.

In one distributed-computing embodiment, different components of derivation system 200 operate on different computing resources. For example, target parameter identifier 210 may operate on a client device, pathway selector 220 and cascade-index selector 230 may operate on a server or cloud resource, calculator 240 may operate on a numerical processing node, and storage or reporting module 270 may operate on a remote database or reporting service. In some embodiments, this distribution improves throughput, scalability, auditability, or integration with other technical systems.

In one proof-linked embodiment, the derivation trace includes one or more references to predetermined Recognition Physics theorem identifiers, proof records, certificate identifiers, or rule identifiers corresponding to one or more selected constants, scale relations, or cascade selections. In some embodiments, such proof-linked references are used to increase auditability or reproducibility. In some embodiments, such proof-linked references are included in a user report, a stored parameter record, or an application programming interface response.

In one extended-parameter example, calculator 240 derives one or more values in addition to the canonical five-parameter family. In some embodiments, the one or more additional values include a bases-per-turn quantity, a scaled coherence quantity, a scaled rate quantity, a stability-related quantity, a coupling-related quantity, a ratio quantity, or another dependent quantity derived from one or more canonical outputs. In some embodiments, the extended parameters are used in downstream ranking, design qualification, model initialization, or validation workflows.

In one acceptance-testing embodiment, one or more candidate systems are screened against one or more threshold conditions derived from the disclosed framework. In some embodiments, a candidate system is screened for compatibility with a selected E_n value. In some embodiments, a candidate system is screened for compatibility with a selected E_bp range or R0 range. In some embodiments, a candidate system is screened for consistency with a selected geometry condition based on X_DNA or P0. In some embodiments, the threshold conditions are used in a laboratory workflow, a software design workflow, or a procurement or synthesis workflow.

In some embodiments, the equations and workflows disclosed herein may be implemented in symbolic form, numerical form, fixed-precision form, arbitrary-precision form, interval-bounded form, or combinations thereof. In some embodiments, constants are retrieved from stored values. In other embodiments, one or more constants are computed from more fundamental constants. In some embodiments, unit conversion occurs during the derivation. In other embodiments, unit conversion occurs only after a canonical value has been generated.

In some embodiments, steps described in connection with one example may be combined with steps described in connection with another example. In some embodiments, one or more modules described in connection with one embodiment may be combined, separated, reordered, duplicated, or omitted in another embodiment. In some embodiments, one or more outputs described in connection with one example may serve as inputs to one or more later derivations, validation processes, design tools, reporting processes, or experimental workflows.

The foregoing examples demonstrate that the disclosed methods, systems, and media can be implemented in a wide variety of practical forms while preserving the central inventive concept of deriving characteristic DNA parameters parameter-free from Recognition Physics principles. The invention is therefore not limited to any single software architecture, reporting format, sequence class, analog class, validation procedure, or downstream engineering workflow.

In some embodiments, derivation system 200 is implemented on a computing device comprising at least one processor, memory storing instructions, and at least one communication interface. The at least one processor executes the instructions to perform at least target parameter identification, pathway selection, pathway-selection value determination, numerical calculation, derivation-trace generation, and output reporting as described herein. In some embodiments, the computing device further comprises at least one data store (local or remote) storing at least one constant library and/or parameter records including derived values and derivation traces, and the computing device exchanges requests and responses via the communication interface.

As used herein, a “module” may be implemented as software instructions stored in memory and executed by one or more processors, as firmware, as dedicated circuitry, or as any combination thereof. Unless expressly stated otherwise, references to modules do not require a specific hardware partition and may be implemented as logical functions executed on one or more computing devices.

In some embodiments, validation module 260 performs at least one acceptance test on a derived numerical value after core derivation. In one example, the system compares a derived value Pd to a reference value Pr (e.g., a literature value, a measured value, or a value from a selected reference record) and computes a percent-difference metric Delta=ABS(Pd−Pr)/Pr.

In one example acceptance rule, the system sets an acceptance flag to PASS when Delta<=T, where T is a predetermined threshold stored in memory and selected according to at least one configuration rule. In some embodiments, T is selected from 0.01 to 0.25. In a default embodiment, T=0.10 for geometric parameters and T=0.15 for energetic parameters. In some embodiments, upon determining PASS or FAIL, the system outputs and/or stores a diagnostic record in the derivation trace identifying the parameter, the reference value Pr, the derived value Pd, the computed Delta, and the selected T.

In another example acceptance rule, the system evaluates an internal consistency metric based on linked outputs, including verifying that P₀=X(DNA)·φ² within a predetermined numerical tolerance and that N(bp_per_turn)=P₀/(X(DNA)/4) within the tolerance, and sets the acceptance flag based on whether the checks pass.

The embodiments described herein are linked by a common inventive concept in which a target parameter identifier, a pathway selector, and a calculator cooperate to derive characteristic parameters from at least one input and to generate a derivation trace identifying intermediate values and constants used to produce an output value. The method, system, and computer-readable medium claim forms correspond to the same underlying derivation pipeline and may be practiced using the same computing implementation.

Worked Example—X_DNA Derivation and Trace. In one example embodiment, the system receives a target parameter identifier indicating a length-type base parameter for a DNA characteristic length X_DNA. The system classifies the target as a length-type base parameter and selects a cascade index from a permitted set according to the rule-based selection architecture.

The system applies the selected cascade index to an anchor quantity according to the cascade-indexing pathway and computes an intermediate scaled quantity. The system then produces the derived numerical value for X_DNA and generates a derivation trace including: the target identifier, the selected pathway class, the selected cascade index, each intermediate quantity, units, and the final output.

In one example, the derivation trace is stored as a versioned parameter record that includes at least: parameter name, pathway class, selected cascade index, constants used, intermediate values, derived value, units, timestamp, and a hash or identifier of the applied rule table version.

Validation Example. In one example, the system computes a percent-difference metric Δ=|P_d−P_r|/P_r relative to a reference value P_r and sets an acceptance flag to PASS when Δ≤T and FAIL otherwise, where T is a configured threshold.

Internal Consistency Example. In one example, the system verifies at least one consistency relation among derived parameters within a tolerance and records the result in the derivation trace.

These example workflows provide a technical implementation in which the derived numerical value is automatically produced, stored with traceability metadata, and used to initialize or constrain downstream computational workflows.

What is claimed is:

1. A computer-implemented method for deriving a characteristic DNA parameter, the method comprising:
receiving, by one or more processors, a target parameter identifier corresponding to a characteristic parameter associated with deoxyribonucleic acid (DNA);
classifying, by the one or more processors, the target parameter identifier into a parameter class;
selecting, by the one or more processors, a derivation pathway based on the parameter class, the derivation pathway comprising cascade indexing, geometric scaling, or efficiency modulation;
determining, by the one or more processors, at least one pathway-selection value comprising a cascade index, a geometric factor, a multiplicity factor, or a structural factor according to a rule-based Recognition Physics selection architecture;
calculating, by the one or more processors, a derived numerical value for the characteristic DNA parameter using (i) at least one universal physical constant, (ii) at least one Recognition Physics constant, and (iii) the at least one pathway-selection value, without using any empirically fitted DNA-specific coefficient as an input to the calculating;
generating, by the one or more processors, a derivation trace identifying at least the selected derivation pathway and the at least one pathway-selection value;
using, by the one or more processors, the derived numerical value to populate, constrain, validate, rank, or filter at least one DNA construct design record, simulation initialization record, sequence screening record, or construct-selection workflow; and
outputting the derived numerical value and the derivation trace in association therewith.

2. A system for deriving a characteristic DNA parameter, the system comprising:
one or more processors; and
memory storing instructions that, when executed by the one or more processors, cause the system to:
receive a target parameter identifier corresponding to a characteristic parameter associated with DNA;
classify the target parameter identifier into a parameter class;
select a derivation pathway based on the parameter class, the derivation pathway comprising cascade indexing, geometric scaling, or efficiency modulation;
determine at least one pathway-selection value according to a rule-based Recognition Physics selection architecture;
calculate a derived numerical value for the characteristic DNA parameter using (i) at least one universal physical constant, (ii) at least one Recognition Physics constant, and (iii) the at least one pathway-selection value, without using any empirically fitted DNA-specific coefficient as an input to the calculation;
generate a derivation trace identifying at least the selected derivation pathway and the at least one pathway-selection value;
populate, constrain, validate, rank, or filter at least one DNA construct design record, simulation initialization record, sequence screening record, or construct-selection workflow using the derived numerical value; and
output the derived numerical value and the derivation trace.

3. A non-transitory computer-readable medium storing instructions that, when executed by one or more processors, cause the one or more processors to:
receive a target parameter identifier corresponding to a characteristic parameter associated with DNA;
classify the target parameter identifier into a parameter class;
select a derivation pathway based on the parameter class, the derivation pathway comprising cascade indexing, geometric scaling, or efficiency modulation;
determine at least one pathway-selection value according to a rule-based Recognition Physics selection architecture;
calculate a derived numerical value for the characteristic DNA parameter using (i) at least one universal physical constant, (ii) at least one Recognition Physics constant, and (iii) the at least one pathway-selection value, without using any empirically fitted DNA-specific coefficient as an input to the calculation;
generate a derivation trace identifying at least the selected derivation pathway and the at least one pathway-selection value;
use the derived numerical value to populate, constrain, validate, rank, or filter at least one DNA construct design record, simulation initialization record, sequence screening record, or construct-selection workflow; and
output the derived numerical value and the derivation trace.

4. The method of claim 1, wherein the target parameter identifier specifies a plurality of canonical DNA parameters comprising a characteristic DNA length parameter X_DNA, a helical pitch parameter P0, a coherence-energy parameter E_coh, a base-pair stabilization parameter E_bp, and a characteristic rate parameter R0, and wherein the method comprises deriving the plurality of canonical DNA parameters during a single execution.

5. The method of claim 4, wherein deriving the characteristic DNA length parameter comprises calculating X_DNA according to:
$$X\_DNA = L\_Planck * ( X\_opt ) ∧ ( - 90 ) , where X\_opt = phi / pi and phi = ( 1 + s q r t ( 5 ) ) / 2.$$

6. The method of claim 5, wherein deriving the helical pitch parameter comprises calculating P0 according to:
$$P 0 = X\_DNA * ( p h i ∧ 2 ) .$$

7. The method of claim 6, further comprising calculating a characteristic bases-per-turn quantity N_bp_per_turn according to:
$$N\_bp \_per \_turn = P 0 / ( X\_DNA / 4 ) = 4 * ( p h i ∧ 2 ) .$$

8. The method of claim 7, wherein deriving the coherence-energy parameter comprises calculating E_coh according to:
$$E\_coh = E\_Planck * ( X\_opt ) ∧ ( 101 ) .$$

9. The method of claim 8, wherein deriving the base-pair stabilization parameter comprises calculating E_bp according to:
$$E\_bp = E\_coh * X\_opt * N\_H ,$$
where N_H is a hydrogen-bond multiplicity parameter.

10. The method of claim 9, wherein the hydrogen-bond multiplicity parameter N_H is sequence-dependent and is calculated according to:
$$N\_H = ( 2 * \#AT + 3 * \#CG ) / ( \#AT + \#CG ) ,$$
where #AT is a number of adenine-thymine base pairs and #CG is a number of cytosine-guanine base pairs in a selected DNA sequence, region, library, or local window.

11. The method of claim 8, further comprising calculating a scaled coherence quantity E_n according to:
$$E\_n = n * E\_coh ,$$
where n is an integer coherence level.

12. The method of claim 11, wherein deriving the characteristic rate parameter comprises calculating R0 according to:
$$R 0 = ( E\_coh / h ) * ( P 0 / ( X\_DNA / 4 ) ) * ( 2 / ( phi ∧ 62 ) ) .$$

13. The method of claim 12, further comprising calculating a scaled rate quantity R according to:
$$R = n * R 0 ,$$
where n is an integer scaling factor.

14. The method of claim 1, wherein the determining of the at least one pathway-selection value comprises:
determining whether the target parameter is a base parameter or a dependent parameter;
when the target parameter is a base parameter, determining whether the target parameter is a length-type base parameter or an energy-type base parameter;
generating a candidate set of cascade indices for the identified parameter type;
evaluating the candidate set against at least one rule reflecting dimensionality, recognition multiplicity, structural class, or stability condition; and
selecting, as the at least one pathway-selection value, a cascade index that is present in a retrieved permitted set obtained by querying the finite stored rule table in memory for the target parameter identifier and the parameter class.

15. The method of claim 14, wherein the target parameter identifier corresponds to a nucleic-acid analog system, and wherein determining the at least one pathway-selection value comprises modifying at least one of dimensionality, recognition multiplicity, structural class, geometric factor, stability criterion, or cascade-index selection rule relative to a canonical DNA embodiment.

16. The method of claim 15, wherein the nucleic-acid analog system comprises ribonucleic acid, peptide nucleic acid, xDNA, or a Hachimoji nucleic-acid system.

17. The system of claim 2, wherein the instructions further cause the system to implement:
a target parameter identifier module;
a pathway selector;
a cascade-index selector;
a calculator;
a derivation trace generator;
a validation module; and
a storage or reporting module.

18. The system of claim 17, wherein the validation module is configured to compare the derived numerical value, after calculation, to at least one reference value and to compute a percent-difference metric Delta=ABS(Pd−Pr)/Pr, to set an acceptance flag to PASS when Delta<=T and to FAIL otherwise, wherein T is a threshold stored in memory and selected from 0.01 to 0.25, and to generate a comparison output comprising at least Pr, Pd, Delta, and T, without using the at least one reference value as an input to the calculation.

19. The non-transitory computer-readable medium of claim 3, wherein the instructions further cause the one or more processors to receive the target parameter identifier through an application programming interface request and to return the derived numerical value and the derivation trace in a machine-readable response.

20. The non-transitory computer-readable medium of claim 19, wherein the instructions further cause the one or more processors to store, in a database or versioned repository, a parameter record comprising a parameter name, a pathway class, one or more constants used, one or more selected pathway-selection values, the derived numerical value, and the derivation trace.
